(19) United States
(12) Patent Application Publication
(10) Pub. No.: US 2015/0328406 A1
(43) Pub. Date: Nov. 19, 2015

(54) HAND-HELD PRE-FILLED SYRINGE ASSEMBLY

(71) Applicant: NEW INJECTION SYSTEMS LTD, Ipswich Suffolk (GB)

(72) Inventor: Stephen Dunne, Ipswich Suffolk (GB)

(21) Appl. No.: 14/442,898

(22) PCT Filed: Nov. 18, 2013
(86) PCT No.: PCT/EP2013/074084

§ 371 Date: May 14, 2015

(30) Foreign Application Priority Data
Nov. 16, 2012 (GB) 1220738.7
Nov. 17, 2012 (GB) 1220750.2
Dec. 13, 2012 (GB) 1222431.7

(51) Int. Cl. A61M 5/31; A61M 5/20; A61M 5/34

(52) U.S. Cl. CPC A61M 5/3134; A61M 5/3135; A61M 5/345; A61M 5/2033; A61M 2005/3103; A61M 2205/15; A61M 2005/311

(57) ABSTRACT

A hand-held pre-filled syringe assembly comprises a syringe barrel, a piston slidably located within the syringe barrel, biasing means coupled to the piston and acting to bias the piston within the syringe barrel, a hypodermic needle, and an openable closure having an inlet and an outlet disposed between the syringe barrel and the hypodermic needle. The inlet of the openable closure is removably coupled to a nozzle of the syringe barrel by a first coupling and the outlet of the openable closure is coupled to a proximal end of the hypodermic needle by a second coupling. The openable closure defines a through-channel extending between the inlet and the outlet that is closed by a normally-closed valve. The syringe assembly contains a liquid medicament retained within the syringe barrel under pressure applied by the piston such that the liquid medicament is delivered through the hypodermic needle when the normally-closed valve is opened.

The invention relates to hand-held pre-filled syringe assemblies having an openable closure disposed between a syringe barrel and a hypodermic needle.

### BACKGROUND

Industry standard pre-filled syringes such as the BD Hypak, the Gerresheimer RTF or ClearJect, the Schott TopPak, the Daikyo Crystal Zenith® Syringe, and other commercially available glass or plastic ready to fill syringes, are commonly used as the primary pack or primary container for auto-injectors.

Pre-filled syringes are filled by manufacturers in controlled environments, eliminating the need for a patient or a medical professional to fill them from vials or ampoules prior to use. Pre-filled syringes typically have life storage of two years or more.

Historically the industry has been reliant upon these well-established off-the-shelf primary containers, usually the glass versions. Most of the alternative auto-injector technologies require a bespoke primary container, which introduces unwanted risk and cost to the development process. However, the standard glass pre-filled syringe, and to a lesser extent the standard plastic pre-filled syringe, presents a number of problems.

Glass pre-filled syringes have a number of disadvantages, which include:
They are fragile and not well suited to use in spring-driven auto-injector devices. A pressure spike or pulse created when the auto-injector spring hits the syringe stopper or piston can cause chipping or breakage of the syringe.
Glass is dimensionally difficult to control during syringe manufacture, so syringe tolerances are broad. This is especially true of the length, making it difficult to design an auto-injector device to fit round it.
The syringe stopper or piston has four functions: drug delivery, oxygen barrier, humidity barrier and sterility barrier. This results in a need for complex multi-ribbed components that form a tight seal with the syringe barrel. This tight seal results in a need to lubricate the inside of the barrel, for example by siliconisation to minimize friction and to prevent the piston or stopper sticking to the barrel during long storage times.
Siliconisation (i.e. treatment with a silicone coating or oil) may can cause stability problems with the drug contained in the pre-filled syringe.
The epoxy glue used in staked needle syringes typically used in auto-injectors can interact with the drug.
Syringe nozzles are typically formed over a tungsten pin. Residue of the tungsten pin can interact with the drug during storage.
The drug contained within a pre-filled syringe is in contact with the needle metal during prolonged storage, which can cause drug stability problems.
The drug is typically in contact with a needle metal during prolonged storage, and this requires a rubber cap, or ‘boot’, to close the opening at the needle tip. Application or removal of the rubber cap can lead to needle damage.

Plastic pre-filled syringes also have a number of disadvantages, which include:
They need to be manufactured in clear plastic with high oxygen barrier properties, which are always inferior to glass.
Because of the high oxygen barrier requirement, plastic pre-filled syringes are expensive relative to glass pre-filled syringes.
Extractables and leachables from the plastic forming the syringe are higher than in glass containers. Extensive testing is required before they can be safely used.
The candidate plastics are not as ‘known’ as glass. This results in an industry reluctance to adopt them.
As with glass pre-filled syringes, the drug contained within the pre-filled syringe is in contact with the needle metal during prolonged storage, which can cause drug stability problems.
The drug is typically in contact with a needle metal during prolonged storage, and this requires a rubber cap, or ‘boot’, to close the opening at the needle tip. Application or removal of the rubber cap can lead to needle damage.

### DESCRIPTION OF THE INVENTION

The invention provides a hand-held pre-filled syringe assembly and an openable closure for a hand-held pre-filled syringe assembly as defined in the appended independent claims, to which reference should now be made. Preferred or advantageous features of the invention are set out in dependent sub-claims.

Thus, a hand-held pre-filled syringe assembly may be provided comprising, a syringe barrel having a nozzle, stopper slidably located within the syringe barrel, a biasing means coupled to the piston and acting to bias the piston towards the nozzle, a hypodermic needle, and an openable closure having an inlet and an outlet disposed between the syringe barrel and the hypodermic needle. The inlet of the openable closure is removably coupled to the nozzle of the syringe barrel by a first coupling and the outlet of the openable closure is coupled to a proximal end of the hypodermic needle by a second coupling. The openable closure defines a through-channel extending between the inlet and the outlet that is closed by a normally-closed valve. The normally-closed valve can be actuated by relative movement of the syringe barrel and a portion of the openable closure, for example the portion of the openable closure that is attached to the needle. The pre-filled syringe assembly contains a liquid medicament retained within the syringe barrel under a pressure applied by the piston, such that the liquid medicament is delivered through the hypodermic needle when the normally-closed valve is opened.

The pre-filled syringe assembly may advantageously be used as an auto-injector or as a component of an auto-injector. An auto-injector consisting of, or comprising, any hand-held pre-filled syringe described herein may also be provided. For example, an auto-injector may be provided by a pre-filled syringe assembly and a housing. The invention may allow the use of conventional pre-filled syringe packaged in an auto-injector with a valve mechanism between the syringe and needle, where the liquid drug contents are maintained under pressure during storage.

The use of a removably couplable openable closure that is disposed between the syringe barrel and a hypodermic needle allows for the use of industry standard pre-filled syringes to create auto-injectors without the need to develop a novel primary pack and get regulatory approval/industry acceptance of the novel primary pack. The pre-filled syringe assembly disclosed herein provides many advantages over the conventional use of industry standard pre-filled syringes in auto-injectors. Thus, the present invention allows for the use of standard primary packs (glass or plastic pre-filled syringes) in auto-injectors with additional advantages not achievable with use of conventional pre-filled syringe assemblies.

A particular advantage of the assembly is the dry needle during storage. The openable closure is disposed between the nozzle of the syringe barrel and the hypodermic needle. Thus, the needle does not contact the liquid medicament retained within the syringe barrel until the closure is opened, thereby eliminating unwanted needle/drug interactions. The ability to maintain a dry needle while using an industry standard syringe barrel is an extremely advantageous feature.

The fact that the needle is kept dry during storage eliminates the need for a needle ‘boot’ to close the needle and retain the liquid medicament. This eliminates the potential for needle damage during application and removal of the ‘boot’. Preferably the pre-filled syringe assembly does not comprise a needle ‘boot’ for sealing the distal end of the hypodermic needle.

The lack of a requirement for a needle boot allows the possibility of using smaller gauge needles, which may be more comfortable to use in certain circumstances.

Conventional pre-filled syringe assemblies, for example as typically used in auto-injectors, do not contain a liquid medicament that is stored under pressure. Thus, the piston or stopper that seals the barrel may move as the liquid and/or air within the syringe barrel expands and contracts. By applying a pressure to the piston that constantly urges the piston towards the nozzle of the syringe barrel, the amount of piston movement may be reduced. This may be a particular advantage during air transport. Reducing the piston movement during air transport may reduce the risk of contamination or loss of sterility. Additionally, because the liquid medicament contents are under positive pressure relative to atmosphere at all times, there is less likelihood of foreign matter entering the sterile environment and contaminating the drug. This is particularly important as drugs formulations for injectables cannot include any preservatives.

The fact that the piston is constantly biased towards the nozzle also provides delivery advantages. The liquid medicament is delivered through the hypodermic needle as soon as the normally-closed valve in the openable closure is opened. In conventional auto-injectors an actuation force, for example provided by a spring, is brought into contact with the stopper to deliver the medicament. This causes a pressure spike or peak which may cause user discomfort and may damage the syringe-barrel. Barrel damage is a particular risk in an auto-injector using industry standard glass syringe primary packaging. Because the liquid is maintained under constant pressure in the present invention, an auto-injector based on the pre-filled syringe assembly may be simplified. For example, there is no need to introduce damper mechanisms to ameliorate the activation pressure pulse. There is also no need to deliberately fill the syringe with an air bubble to minimize the activation pressure pulse.

A constant pressurisation of the liquid medicament advantageously provides for automatic leak detection. It is important to know whether a medicament has leaked, as any leak may be a site of contamination. Further, a leaked medicament may not provide a patient with a full required dose. Where the liquid is under constant pressure during storage, due to the piston being biased towards the nozzle, any leak will result in the liquid medicament being expelled from the assembly and detected by causing the piston to visibly move to a non full dose position thus alerting the user.

Because industry standard pre-filled syringes can be used, rather than bespoke primary containers, the syringe barrels may be filled using conventional filling lines. This is likely to improve industry acceptance of the pre-filled syringes.

Advantageously, the normally-closed valve is of the aerosol valve type. Such valves are well known and understood. Preferably the aerosol-valve is a springless valve in order to minimise the number of components or materials in contact with the liquid medicament and reduce the risk of unwanted drug/material interactions. Alternatively, the aerosol-valve may comprise a polymeric spring so that a metal is not arranged in contact with the liquid medicament during storage. Preferably, the entire openable closure is formed entirely of polymeric materials. It is preferred that the normally-closed valve is not a pierceable septum.

The openable closure may comprise a locking means for retaining the normally-closed valve in an open position after actuation. This allows the full volume of liquid medicament to be expelled from the syringe barrel once the normally-closed valve has been opened. In other embodiments it may be desirable to shut off the delivery of liquid medicament at a point during delivery by allowing the normally-closed valve to close.

Preferably, the first coupling is a luer-type coupling, for example a luer lock or a luer slip. Such connections are commonly used on industry standard syringes, and the use of such couplings may allow any standard syringe assembly to be converted into a syringe assembly according to the present invention. Where staked needles are used in glass pre-filled syringes, the nozzle of the syringe barrel needs to have a narrow bore. Such narrow bores can only be produced by forming the nozzle over a tungsten pin. As stated above, tungsten contamination is a major problem for some medicaments. Syringe barrels have nozzles intended to be coupled using luer locks do not need to be formed over tungsten rods, and the tungsten contamination issue is advantageously avoided.

It may be advantageous if the second coupling is also a luer-type coupling, for example a luer lock or a luer slip. This would allow a hypodermic needle to be removably couplable to the assembly. Alternatively, the openable closure may be affixed to a hypodermic needle. For example, the openable closure may comprise a staked needle.

The piston may be coupled to a plunger for sliding the piston within the syringe barrel. The piston may then be coupled to the biasing means.

Preferably the biasing means is a spring, for example a helical spring. The spring preferably acts on the stopper or piston, either directly or via a connection component, to bias the piston towards the nozzle and pressuring the liquid. The spring may be retained within the syringe barrel by a cap or seal spanning a proximal end of the syringe barrel. The cap may include a recess to accommodate a spring. The cap or seal is connected to walls of the syringe barrel such that the spring biases the piston relative to the syringe barrel. A cap or seal retaining a spring may be termed a spring lock.

Preferably, a cap seals the syringe barrel against oxygen and/or humidity. This means that the piston does not need to perform these functions. For example, if the spring is contained within the syringe barrel then the spring lock may form a seal with the syringe barrel and the piston need not be an oxygen barrier. This may allow reduction or elimination in the silicone oil lubrication of the barrel, which can interact with some drug active ingredients especially biologics. This possibility is due to the availability of low oxygen barrier/self lubricating materials, such as PTFE and silicone, which may be used as the piston. Thus, the syringe barrel may not be siliconised and may be a barrel that does not comprise silicone lubricant. The cap or spring lock is not in contact with the liquid drug and so can be manufactured in a large range of materials.

An openable closure for a hand-held pre-filled syringe assembly may also be provided. The openable closure may define an inlet having a first coupling for removably coupling the inlet to a nozzle of a syringe barrel, and an outlet having a second coupling for connection to a hypodermic needle. The openable closure may further comprise a channel defining a fluid flow path extending through the openable closure between the inlet and the outlet, the channel being closable by a normally-closed valve that can be actuated by relative movement of the syringe barrel and the hypodermic needle to open the channel such that liquid can flow through the channel between the inlet and the outlet. In use, the openable closure may be coupled to a syringe barrel of a pre-filled syringe and a hypodermic needle to allow a liquid medicament to be retained under pressure within the syringe barrel in isolation from the hypodermic needle until actuation of the normally-closed valve.

Preferably, the normally-closed valve is an aerosol valve, for example in which the aerosol valve is a springless valve or in which the aerosol valve comprises a polymeric spring, preferably in which the normally-closed valve is constructed entirely of polymeric materials.

Advantageously, the normally-closed valve may comprise a locking means for retaining the normally-closed valve in an open position after actuation.

The first coupling may be a luer-type coupling, for example a luer lock or a luer slip, and/or the second coupling may be a luer-type coupling, for example a luer lock or a luer slip.

The openable closure may comprise a hypodermic needle affixed to the openable closure at the second coupling, for example an openable closure according to any preceding claim comprising a staked hypodermic needle.

An auto-injector may be provided comprising an openable closure as described herein.

### SPECIFIC EMBODIMENTS OF THE INVENTION WILL NOW BE DESCRIBED WITH REFERENCE TO THE FIGURES, IN WHICH

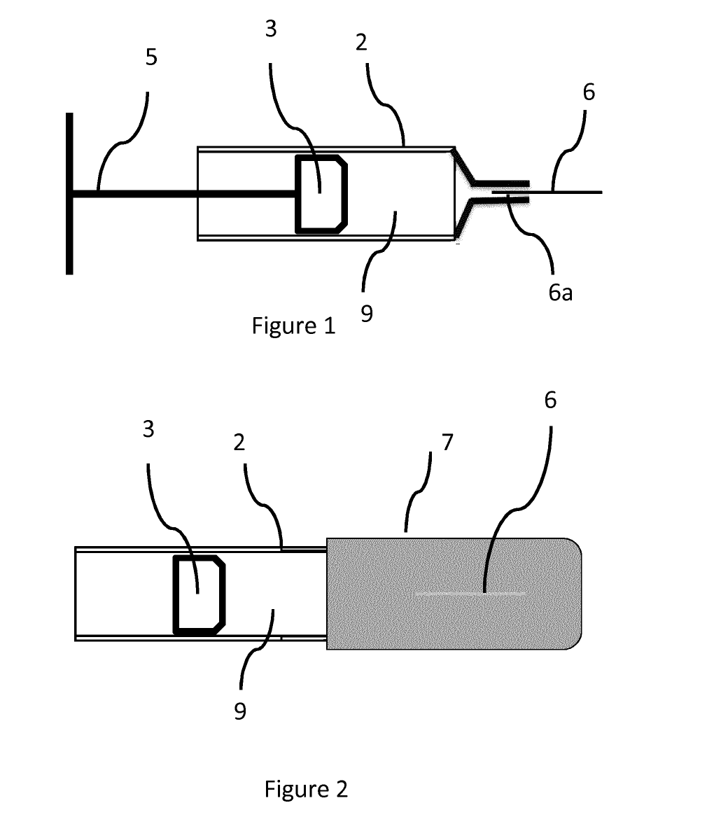

FIGS. 1 and 2 illustrate state of the art syringe assemblies;

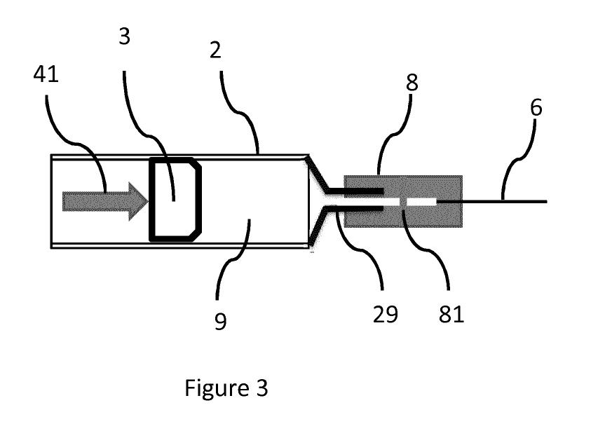

FIG. 3 is a schematic illustration showing a pre-filled syringe assembly embodying the invention;

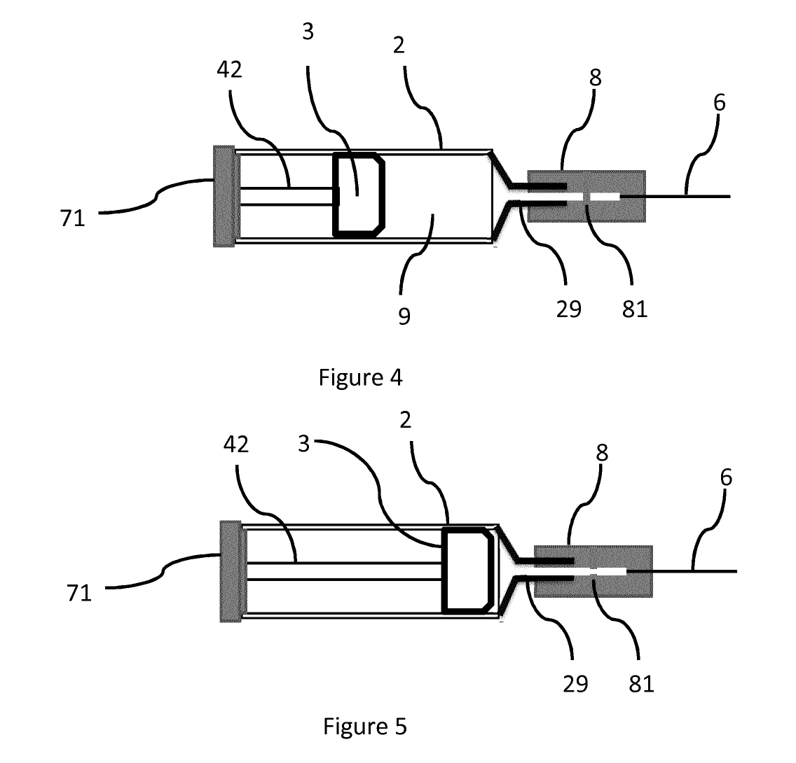

FIGS. 4 and 5 are schematic illustrations of a specific embodiment of a pre-filled syringe assembly embodying the invention,

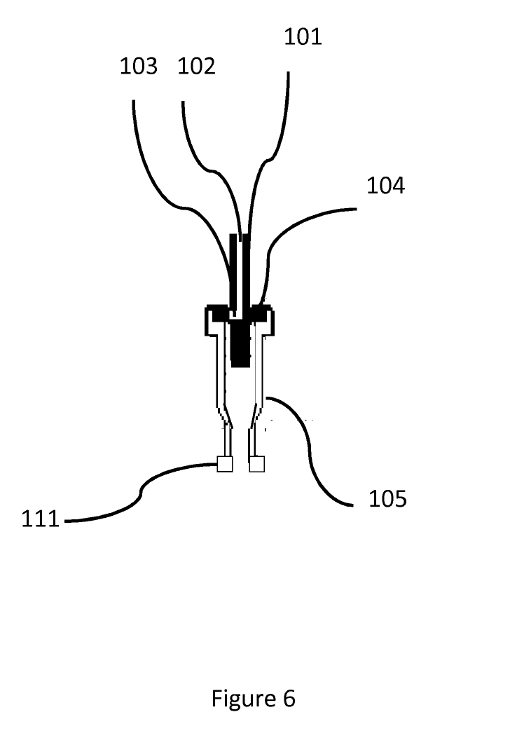

FIG. 6 illustrates an openable closure according to an aspect of the invention and suitable for use as a component of a pre-filled syringe assembly embodying the invention;

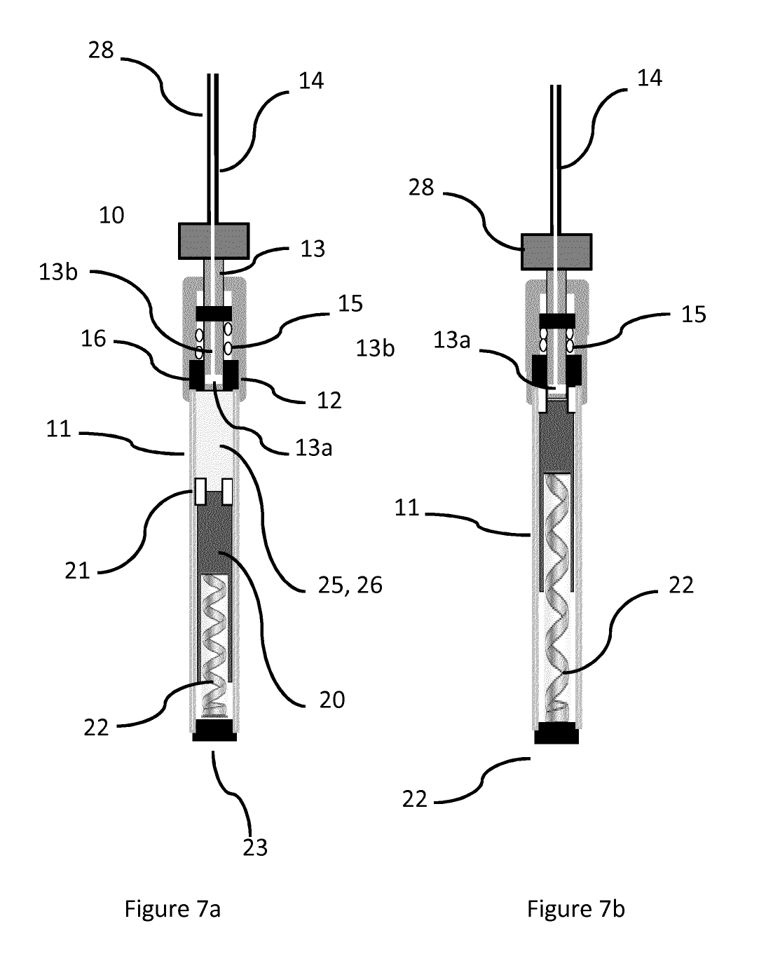

FIGS. 7 to 12 illustrate various specific embodiments of pre-filled syringes and auto-injectors embodying the invention.

In FIG. 1 a state of the art pre-filled syringe is schematically illustrated. A syringe barrel 2 has within a piston or-stopper 3, a plunger rod 5, and a needle 6. The needle 6 is glued into place with an adhesive 6a in the case of glass barrels, and insert molded in the case of plastic barrels. The barrel 2 holds a liquid medicament 9.

FIG. 2 also shows a state of the art of a pre-filled syringe as typically used with an auto-injector. The needle 6 is covered by a sheath 7 also called a ‘boot’ which has a soft rubber or elastomer portion that prevents evaporation of liquid drug contents 9 via the needle 6, and also protects drug contents 9 from oxygen contamination. The sheath or cap or boot 7 is sometimes difficult to remove as it needs to be fitted tightly to the needle and syringe barrel. The soft elastomer can also damage the Needle 6 when the Needle is pushed into it for a tight seal.

The piston or stopper which seals the liquid medicament 9 from the atmosphere has no plunger rod, and such syringes are used in auto-injectors where a spring forces the piston 3 to move and expel the liquid medicament via the needle 6. The liquid medicament contents 9 are kept at atmospheric pressure during storage and up to the time of use.

The liquid medicament contents 9 are bounded by the syringe barrel 2 walls, the piston 3, the needle 6 and the needle boot 7. In conventional devices the contents 9 are kept at atmospheric pressure up to the point of injection. Additionally the contents 9 also usually have a bubble of air trapped within. This bubble is often deliberately introduced to dampen the system and to minimize pressure pulses when the spring is released at the point of use. During air transport this bubble can double in size pushing the piston 3 out and back again. This can lead to loss of sterility of the pre-filled syringe assembly.

Because the contents 9 can be at various times at a lower pressure than the surrounding environment the drug contents 9 are susceptible to contamination via a faulty piston 3 or faulty boot 7.

A pre-filled syringe assembly according to the present invention may advantageously minimise or eliminate these problems.

FIG. 3 schematically illustrates a hand-held pre-filled syringe according to an embodiment of the invention. The syringe assembly includes a syringe barrel 2 with a piston 3 slidably disposed within the barrel, and a hypodermic needle 6. An openable closure 8 is disposed between a nozzle 29 of the syringe barrel 2 and the needle. The openable closure 8 comprises a normally-closed valve 81 that is actuatable to allow liquid medicament to pass. The openable closure keeps the needle 6 dry during storage as a liquid medicament 9 contained within the syringe barrel is not in contact with the needle 6 when the closure 8 is closed. This eliminates the need to close the needle tip with an elastomer boot, and so prevents damage to the needle. Additionally the dry needle can be an advantage for many drugs, especially biologics. The normally-closed valve 81 maybe of the aerosol type. The openable closure is fitted to a conventional glass or plastic pre-filled syringe barrel 2 by a suitable luer type connection. A biasing means 41, such as a spring, acts to urge the piston 3 towards the nozzle 29. The biasing means 41 causes the piston to apply pressure to the liquid medicament, which is retained under pressure. The normally closed-valve is openable by relating movement of the needle 6 and the syringe barrel 2. When the normally-closed valve is opened, the pressurised liquid medicament 9 is delivered through the needle 6.

FIGS. 4 and 5 illustrate the use of a specific embodiment of a pre-filled syringe assembly as an auto-injector. With reference to FIG. 4, the pre-filled syringe assembly is the same as described above in relation to FIG. 3, with the exception that a proximal end of the syringe barrel is closed by a cap or spring-lock 71. A helical spring 42 is located in a compressed condition within the syringe barrel 2, acting on the cap 71 and the piston 3. The cap is connected to the syringe barrel, and provides an oxygen and humidity seal. Thus, the piston is urged towards the nozzle 29, and the liquid medicament 9 retained within the syringe barrel is retained under pressure.

In order to deliver the medicament, a user inserts the needle 6 into their skin. Once the needle is at the correct depth, the normally-closed valve 81 is opened. As can be seen from FIG. 5, the opening of the normally-closed valve allows the liquid medicament to be delivered through the needle. This in turn allows the piston to move towards the nozzle, under the biasing influence of the spring 42, thereby delivering the remaining medicament through the needle.

FIG. 6 illustrates an openable closure comprising a normally-closed valve of the aerosol type that may be used in pre-filled syringe assemblies and auto-injectors according to embodiments of the invention. The openable closure has a housing 105, a valve stem 101 with passageway 102 and cross-hole 103, which is closed by gasket 104 when, as shown, the valve is in its normal, closed, position. The illustrated normally-closed valve has no spring, so when opened it doesn't automatically close (although an aerosol-valve having a spring may be advantageous in certain embodiments). The lack of spring in the normally-closed valve provides the advantage that the valve won't necessarily close when the user releases pressure on the skin when used as an auto-injector. It also means that the liquid medicament stored in the syringe barrel is not stored in contact with a spring. An openable closure consisting of, or comprising, this type of normally-closed valve may be connected in various ways to the nozzle of a conventional syringe, for example via a luer connection. For example, the openable closure illustrated in FIG. 6 has a flange 111 to connect to the nozzle of a syringe barrel with a luer lock connection.

In FIGS. 7a and 7b an auto-injector 10 is schematically illustrated, before use in FIG. 7a and after use in FIG. 7b. A conventional pre-filled syringe has a transparent barrel 11 and a piston or stopper 20 with a seal(s) or ribs 21, defining a space 25 inside of which is a liquid medicament 26 is retained. A nozzle of the syringe barrel 11 is attached to an openable closure 12, which includes a normally-closed valve. The openable closure comprises a stem 13 with a stem orifice 13a and stem channel 13b, which is connected to a needle 14. A valve spring 15 holds the stem orifice sealed to a gasket 16. A helical spring 22, partially located by the piston 20, is retained within the syringe barrel 11 by a spring stopper or lock 23. The piston 20 compresses the liquid medicament 26.

For high oxygen and humidity barrier properties the barrel 11 may be made of glass and the spring lock or stopper 23 may form an oxygen and humidity barrier with the barrel 11. In this way the piston or stopper 20, 21 does not need to be a oxygen and humidity barrier, which allows for a greater selection of drug compatible materials having improved properties to be used as the piston. This also allows for the elimination or reduction of silicone lubricant in the barrel 11, which can interact with some biopharmaceutical drugs. For example silicone or PTFE stoppers or part silicone or PTFE stoppers may be used. The piston 20, 21 for instance may be moulded in PTFE with an inner elastomeric not in contact with the drug or the barrel or both.

The barrel 11 may have a luer lock connection on which the openable closure 12 with a luer lock connection may be attached. Alternatively the openable closure 12 may be attached and glued to a Luer slip connection on the barrel 11, preferably with the glue not in contact with the liquid drug contents. Syringe barrels 11 with luer connections have larger bore outlets than syringes with staked needles, allowing for the use of non-tungsten pins in their manufacture. Tungsten can interact un-favorably with some drugs.

Alternatively the outlet bore of the barrel 11 may be sleeved with a plastic or other material to minimize its dead volume (drug left behind after injection) and protect the drug contents from any tungsten or other material used in the barrel manufacture.

The needle 14 may be a safety needle to protect users from needle stick injury. This may be for instance the West Pharmaceuticals NovaGuard, the TipTop or any other safety needle system or device. This addition converts the device into a complete, but simple, auto-injector. Alternatively any such needle protection arrangement may be added to the device in the form of a needle shield or other device.

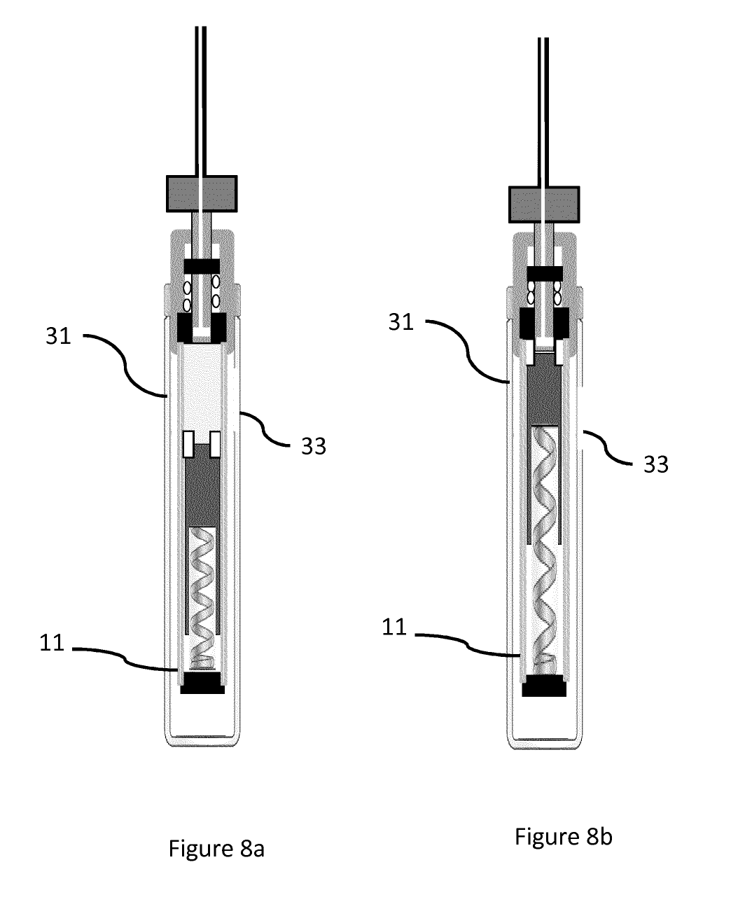

In FIGS. 8a and 8b a pre-filled syringe assembly according to an embodiment of the invention is located within an outer case 31 to form an auto-injector. This may provide a safety feature in case the barrel 11 breaks. The outer case 31 may have viewing ports 33 if it is not transparent. Viewing ports 33 may be open if the barrel 11 is made of glass or other oxygen barrier material and the spring lock 23 is oxygen tight. In FIGS. 8a and 8b the outer case 31 may be made of plastic, glass or metal such as aluminium with viewing ports 33 sealed with aluminium laminate or with glass viewing ports.

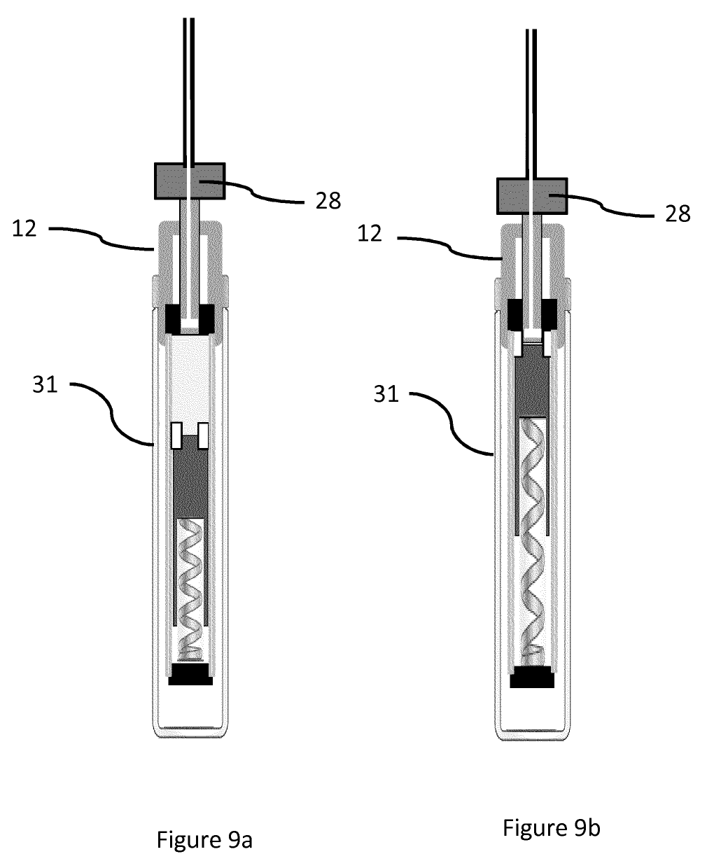

FIGS. 9a and 9b illustrate an auto-injector similar to that shown in FIGS. 8a and 8b, except that the openable closure 12 has no spring. In this case once open the closure 12 stays open even when pressure on skin pad 28 is released.

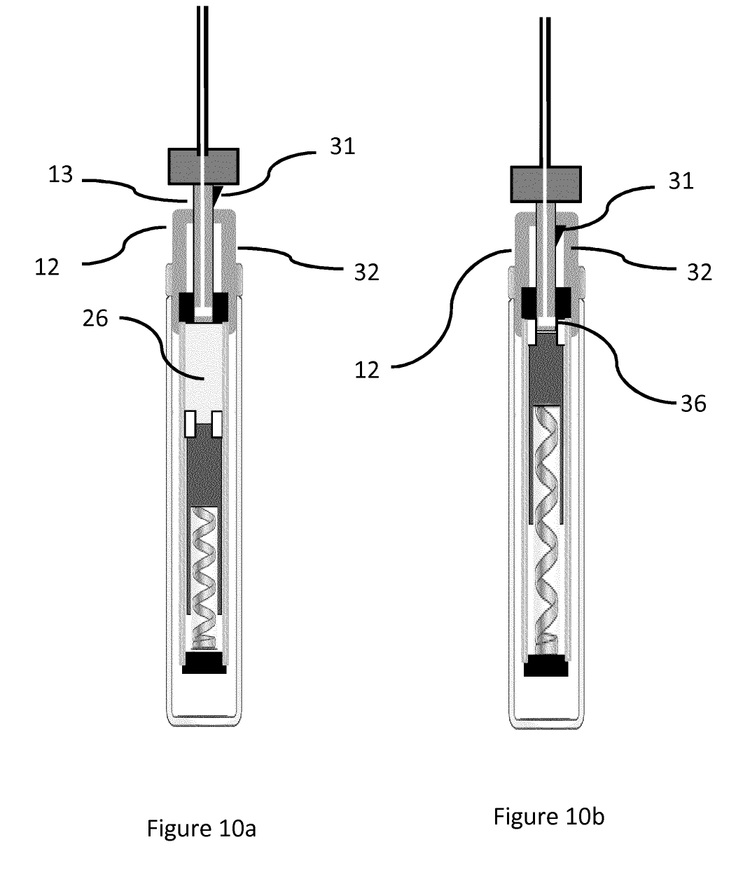

FIGS. 10a and 10b illustrate an auto-injector similar to that shown in FIGS. 9a and 9b. The difference is that the openable closure 12 has a body 32, a stem 13, and a latch 31 attached to the stem 13. When the closure is opened and the cross-hole 36 is in communication with the pressurized liquid medicament 26, the latch 31 on the stem 13 is locked behind the body 32, preventing valve closure due to pressure of the liquid drug 26.

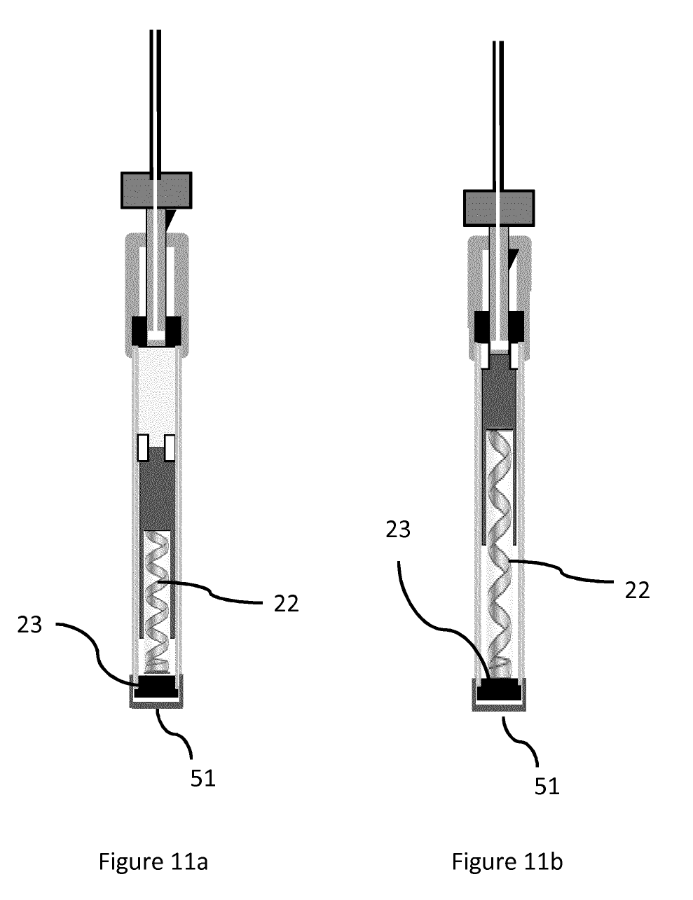

FIGS. 11a and 11b illustrate an auto-injector similar to that shown in FIGS. 10a and 10b, with the difference that the spring lock 23 is crimped and held into place by a ferrule 51. Standard pre-filled syringes have a flange at this point, which may be used to crimp the ferrule on to. The ferrule 51 may be made of any material but is preferably a soft metal such as aluminium alloy so that it can be crimped into place, as is commonly done with vial stoppers, creating a strong anchor point for a spring 22 and, if laminated, excellent oxygen and humidity barrier properties.

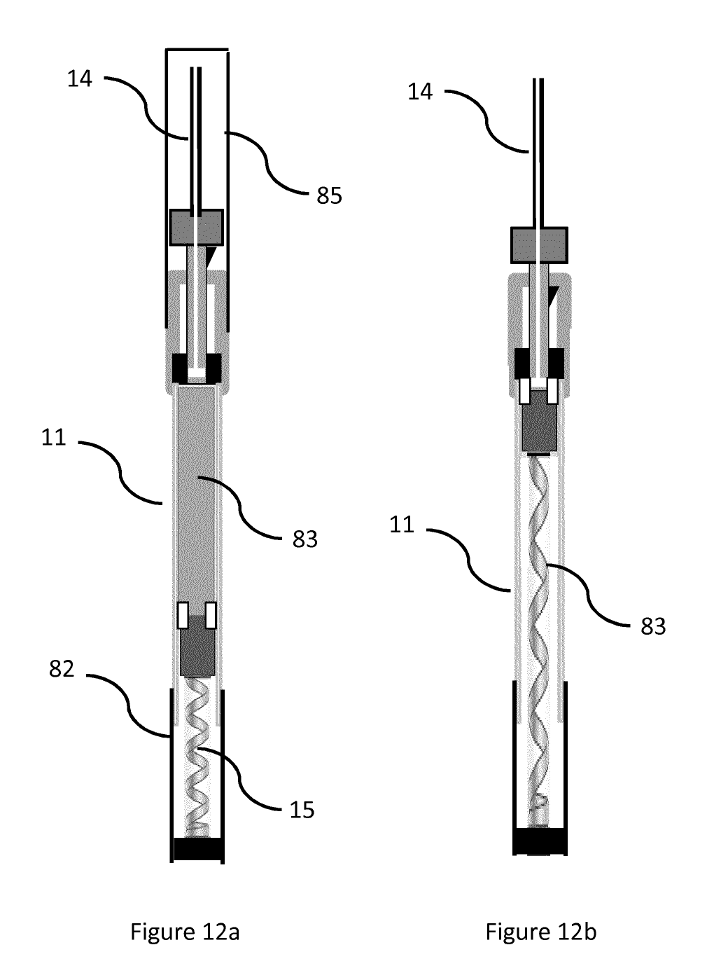

FIGS. 12a and 12b illustrate a pre-filled syringe assembly in which the syringe barrel 11 has a spring 15 held within the barrel 11 by a spring holder 82. The spring holder provides an extension of the space within the syringe barrel. In this embodiment, liquid medicament contents 83 retained within the barrel 11 can be of larger volume than if the spring was also entirely located within the barrel 11, as illustrated in other specific embodiments. A cap 85 protects and keeps the needle 14 sterile before use. The cap 85 is at no time in contact with the needle 14, allowing for smaller than usual needle gauge, such as gauge 29 and 30 and 31 or even smaller needles, and so-called thin walled needles to be used without damage.

A number of combinations of syringe barrel, piston, spring lock and outer case are possible, each providing different oxygen and humidity barrier properties. This is summarized in Table 1.

TABLE 1

| | Low | High | Medium | High | High | High |
|---|---|---|---|---|---|---|
| | Oxygen/ | Oxygen/ | Oxygen/ | Oxygen/ | Oxygen/ | Oxygen/ |
| | humidity | humidity | humidity | humidity | humidity | humidity |
| Part | protection | protection | protection | protection | protection | protection |
| Piston | Conventional | Conventional | Conventional | Conventional | Thermoplastic | Conventional |
| Seal or | | | | | seal (low barrier) | |
| Stopper | | | | | | |
| Barrel | Plastic | Plastic | Glass | Glass | Glass | Glass |
| Spring lock | Open | Open | Open | Oxygen | Oxygen | Open |
| | | | | Barrier | Barrier | |
| Outer case | None or plastic | Metal with | Transparent | Transparent | Transparent | Metal with |
| | | sealed viewing | plastic or opaque | plastic or opaque | plastic or | sealed |
| | | ports or Glass | with holes | holes | opaque with | viewing |
| | | | | | holes | ports |

The syringe barrel in any of the specific embodiments described above may be an industry standard syringe such as a BD Hypak, a Gerresheimer RTF or ClearJect, a Schott TopPak, a Daikyo Crystal Zenith® Syringe or any other commercially available glass or plastic ready to fill syringe with a luer lock or luer slip or cone connection or any other type of ready to fill syringe. The openable closure of any specific embodiment described above may comprise a continuous aerosol type valve, with or without an internal spring, and optionally with a ferrule and gasket that can be crimped to an outer case 31 forming, if needed, an oxygen and humidity barrier.

Any of the pre-filled syringe assemblies described above may be converted into an auto-injector with manual needle insertion and retraction, or auto needle insertion and retraction, or any combination thereof.

Any of the pre-filled syringe assemblies described above may be converted into an auto-injector or patch pump with manual needle insertion and retraction, or auto needle insertion and retraction, or any combination thereof. Any of the pre-filled syringe assemblies described above may be used in conjunction with any liquid medicament, whether a solution or a suspension or a mixture of these, of any viscosity and density. For instance any of the drugs listed below may be injected using the invention:

17-alpha hydroxyprogesterone caproate, Corticotropin (ACTH), Laronidase, Factor VIII, Von Willebrand Factor Complex, Alefacept, Apomorphine Hydrochloride, Darbepoetin Alfa, Nelarabine, Bevacizumab, Interferon beta-1a, 11 mcg, Interferon beta-1a, 33 mcg, Factor IX complex, Interferon beta-1b, Ibandronate Sodium, Botulinum Toxin, Protein C Concentrate, Alglucerase, Imiglucerase, Injection, Secretin, Synthetic, Human, 1 Microgram, Glatiramer actate, Decitabine, Desmopressin acetate, Idursulfase, Etanercept, Epoetin alfa, Anadalufungin, Cetuximab, Ethanolamine Oleate, Hyaluronic acid derivatives, Agalsidase beta, Factor IX non-recombinant, Factor IX recombinant, Factor VIII (human), Factor VIII (porcine), Factor VIII recombinant, Feiba VH, Immune globulin (intravenous) (IVIG), Enfuvirtide, Immune globulin (intravenous) (IVIG), Somatropin, Hepatitis B Immune, Globulin (intravenous) (IVIG), Trastuzumab, von Willebrand factor complex, Adalimumab, Insulin for administration through DME (i.e., insulin pump), Hyaluronic acid derivatives, Mecasermin, Gefitinib, Levoleucovorin calcium, Ranibizumab Injection, Pegaptnib, Urofollitropin, Micafungin, Botulinum toxin type B, Aglucosidase alfa, Galsulfase, Somatropin, Factor Vila, Atacept, Hyaluronic acid derivatives, Hyaluronan derivative, Immune globulin (intravenous) (IVIG), Hemin, Peginterferon alfa-2a, Peginterferon alfa-2b, Epoetin alfa, Somatrem, Efalizumab, Interferon beta-1a, subq, Zoledronic Acid, Infliximab, Treprostinil, Fluocinolone acetonide, intravitreal implant, Zidovudine, Eculizumab, Lanreotide, Histrelin implant, Palivizumab, Hyaluronic acid derivatives, Temozolomide, Antithrombin III (Human), Natalizumab
Panitumumab, Immune globulin (intravenous) (IVIG), Azacitidine, Verteporfin Hyaluronidase, Bovine, Preservative Free, Naltrexone Depot, Teniposide, Omalizumab, 90Y-Ibritumomab tiuxetan, ADEPT, Aldesleukin, Alemtuzumab, Bevacizumab, Bortezomib, Cetuximab, Dasatinib, Erlotinib, Gefitinib, Gemtuzumab,
Imatinib, Interferon alpha, Interleukin-2, Iodine 131 tositumomab, Lapatinib, Lenalidomide, Panitumumab, Rituximab, Sorafenib, Sunitinib, Thalidomide, Trastuzumab;

Plus other biologics or small molecule drugs including a wide range of medicinal products such as vaccines, blood and blood components, allergenics, somatic cells, gene therapy, tissues, and recombinant therapeutic proteins, and substances that are (nearly) identical to the body's own key signalling proteins may also be injected using the invention. Examples are the blood-production stimulating protein erythropoetin, or the growth-stimulating hormone named (simply) “growth hormone” or biosynthetic human insulin and its analogues.

Plus monoclonal antibodies. These are similar to the antibodies that the human immune system uses to fight off bacteria and viruses, but they are “custom-designed” (using hybridoma technology or other methods) and can therefore be made specifically to counteract or block any given substance in the body, or to target any specific cell type.

Plus Receptor constructs (fusion proteins), usually based on a naturally-occurring receptor linked to the immunoglobulin frame. In this case, the receptor provides the construct with detailed specificity, whereas the immunoglobulin-structure imparts stability and other useful features in terms of pharmacology.

Plus any of the following:

Alpha1-Adrenergic Antagonists, Analgesic Agents, Anesthetics, Angiotensin Antagonists, Inflammatory Agents, Antiarrhythmics, Anticholinergics, Anticoagulants, Anticonvulsants, Antidiarrheal Agents, Antineoplastics and Antimetabolites, Antineoplastics and Antimetabolites, Antiplasticity Agents, Beta-Adrenergic Antagonists, Bisphosphonates, Bronchodilators, Cardiac Inotropes, Cardiovascular Agents Central Acting Alpha2-stimulants, Contrast Agents, Converting Enzyme Inhibitors, Dermatologics, Diuretics, Drugs for Erectile Dysfunction, Drugs of Abuse, Endothelin Antegonists, Hormonal Agents and Cytokines, Hypoglycemic Agents

### Hypouricemic Agents and Drugs Used For Gout, Immunosuppressants, Lipid Lowering Agents, Psychotherapeutic Agents, Renin Inhibitors, Serotonergic Antagonist Steroids, Sympathomimetics, Thyroid and Antithyroid Agents, Vasodilators, Vasopeptidase Inhibitor

Or any other drug not listed above capable of being injected and available at present or being developed by any pharmaceutical company or any other company anywhere in the world.

1. A hand-held pre-filled syringe assembly comprising,
a syringe barrel having a nozzle,
a piston slidably located within the syringe barrel,
biasing means coupled to the piston and acting to bias the piston towards the nozzle,
a hypodermic needle, and
an openable closure having an inlet and an outlet disposed between the syringe barrel and the hypodermic needle, in which
the inlet of the openable closure is removably coupled to the nozzle of the syringe barrel by a first coupling and the outlet of the openable closure is coupled to a proximal end of the hypodermic needle by a second coupling, the openable closure defining a through-channel extending between the inlet and the outlet that is closed by a normally-closed valve that can be actuated by relative movement of the syringe barrel and a portion of the openable closure, in which the syringe assembly contains a liquid medicament retained within the syringe barrel under pressure applied by the piston such that the liquid medicament is delivered through the hypodermic needle when the normally-closed valve is opened.

2. A hand-held pre-filled syringe assembly according to claim 1 in which the normally-closed valve is an aerosol valve.

3. A hand-held pre-filled syringe assembly according to claim 2 in which the aerosol valve is a springless valve.

4. A hand-held pre-filled syringe assembly according to claim 2 in which the aerosol valve comprises a polymeric spring.

5. A hand-held pre-filled syringe assembly according to any preceding claim in which the openable closure is constructed entirely of polymeric materials.

6. A hand-held pre-filled syringe assembly according any preceding claim in which the openable closure comprises a locking means for retaining the normally-closed valve in an open position after actuation.

7. A hand-held pre-filled syringe assembly according to any preceding claim in which the first coupling is a luer-type coupling, for example a luer lock or a luer slip.

8. A hand-held pre-filled syringe assembly according to any preceding claim in which the second coupling is a luer-type coupling, for example a luer lock or a luer slip.

9. A hand-held pre-filled syringe assembly according to any of claims 1 to 7 comprising a hypodermic needle affixed to the openable closure at the second coupling, for example a hand-held pre-filled syringe assembly a staked hypodermic needle affixed to the openable closure.

10. A pre-filled syringe assembly according to any preceding claim in which the syringe barrel is a standard pre-filled syringe barrel.

11. A pre-filled syringe assembly according to any preceding claim in which the piston is coupled to a plunger for sliding the piston within the syringe barrel.

12. A pre-filled syringe assembly according to any preceding claim in which the piston is coupled to a spring for biasing the piston towards the nozzle.

13. A pre-filled syringe assembly according to claim 12 further comprising a cap for sealing a proximal end of the syringe barrel, the spring being retained between the cap and the piston to exert a force urging the piston towards the nozzle.

14. A pre-filled syringe assembly according to claim 13 in which the cap seals the syringe barrel against oxygen and/or humidity.

15. An autoinjector comprising a pre-filled syringe assembly according to any preceding claim and a housing.

16. An openable closure for a hand-held pre-filled syringe assembly, the openable closure defining an inlet having a first coupling for removably coupling the inlet to a nozzle of a syringe barrel,
an outlet having a second coupling for connection to a hypodermic needle, and
a channel defining a fluid flow path extending through the openable closure between the inlet and the outlet, the channel being closable by a normally-closed valve that can be actuated by relative movement of the syringe barrel and a portion of the openable closure to open the channel such that liquid can flow through the channel between the inlet and the outlet, the openable closure, in use, being coupled to a syringe barrel of a pre-filled syringe and a hypodermic needle to allow a liquid medicament to be retained under pressure within the syringe barrel in isolation from the hypodermic needle until actuation of the normally-closed valve.

17. An openable closure according to claim 1 in which the normally-closed valve is an aerosol valve, for example in which the aerosol valve is a springless valve or in which the aerosol valve comprises a polymeric spring, preferably in which the normally-closed valve is constructed entirely of polymeric materials.

18. An openable closure according any preceding claim comprising a locking means for retaining the normally-closed valve in an open position after actuation.

19. An openable closure according to any preceding claim in which the first coupling is a luer-type coupling, for example a luer lock or a luer slip, and/or in which the second coupling is a luer-type coupling, for example a luer lock or a luer slip.

20. An openable closure according to any preceding claim comprising a hypodermic needle affixed to the openable closure at the second coupling, for example an openable closure according to any preceding claim comprising a staked hypodermic needle.
